(19) United States
(12) Patent Application Publication
(10) Pub. No.: US 2021/0170377 A1
(43) Pub. Date: Jun. 10, 2021

(54) HETEROATOM SUBSTITUTED ZEOLITES

(71) Applicant: The Board of Trustees of the University of Illinois, Urbana, IL (US)

(72) Inventors: David William Flaherty, Urbana, IL (US); Daniel T. Bregante, Urbana, IL (US)

(73) Assignee: The Board of Trustees of the University of Illinois, Urbana, IL (US)

(21) Appl. No.: 17/112,537

(22) Filed: Dec. 4, 2020

Related U.S. Application Data
(60) Provisional application No. 62/944,412, filed on Dec. 6, 2019.

(51) Int. Cl. B01J 29/89; C01B 39/06; C01B 39/20; B01J 35/10; B01J 37/30; B01J 37/00; B01J 37/06; C07D 301/12

(52) U.S. Cl. CPC B01J 29/89; C01B 39/065; C01B 39/20; B01J 35/1061; B01J 35/1057; B01J 37/30; B01J 2229/37; B01J 37/06; C07D 301/12; C01P 2006/16; B01J 2229/16; B01J 2229/183; B01J 37/009

(57) ABSTRACT

The invention provides methods for completely removing aluminum from existing zeolite frameworks that have been previously considered unalterable due to their small pore sizes and stable crystal structures. Consequently, new combinations of metal atoms and zeolite structures can now be made using the methods disclosed herein. Metal atoms that have useful properties for catalysis and adsorption have been integrated into zeolite structures that provide advantageous size selection or solvation properties to increase rates, conversions, and yields of catalytic processes. The disclosed catalysts and methods reduce the cost of synthesizing useful materials and zeolite structures with compositions of matter that have not been reported.

### RELATED APPLICATIONS

This application claims priority under 35 U.S.C. § 119(e) to U.S. Provisional Patent Application No. 62/944,412, filed Dec. 6, 2019, which is incorporated herein by reference.

### GOVERNMENT SUPPORT

This invention was made with government support under Grant No. W911NF-18-1-0100 awarded by the U.S. Army Research Office. The government has certain rights in the invention.

### BACKGROUND OF THE INVENTION

Decades of research devoted to zeolite and zeotype catalysts have focused on understanding how the unique pore structure of these materials enables size- and shape-selective catalysis. The rates and selectivities toward specific, desired catalytic transformations depends critically on the dispersive interactions between the micropores of the zeolite catalyst and the reactants contained within these spaces. These interactions provide a basis to modify rates and selectivities by stabilizing surface intermediates and transition states along reaction coordinates.

Zeolite catalysts that contain framework-substituted Lewis acidic heteroatoms (e.g., Ti, Sn, Nb, Ta) catalyse stereoselective mono- and di-saccharide isomerization, aldol condensation, alcohol dehydration, Baeyer-Villiger oxidation, and alkene epoxidation reactions. For example, the discovery that Sn-substituted zeolite *BEA (Sn-BEA) activates ketones for Baeyer-Villiger oxidation with hydrogen peroxide (H₂O₂) catalysed a renaissance of work with Sn-BEA for use in other reactions (e.g., glucose isomerization, ethanol dehydration). Titanium-substituted silicalite-1 (TS-1) was developed in the 1980's and has found industrial application as a catalyst for propylene epoxidation with H₂O₂, which inspired the development of subsequent design principles for epoxidation reactions. The presence of Brønsted acid sites (e.g., those formed at framework Al or B atoms)) catalyze secondary reactions that are detrimental to the desired chemistry, even when Al atoms remain in trace quantities (Si:Al=10-150) (J. Catal., 2018, 368, 145). In the context of alkene epoxidation, the desired epoxides formed by primary reaction pathways readily undergo undesirable ring opening hydrolysis over Brønsted acids, which decreases yields. Therefore, researchers invested significant effort into developing synthetic methods that avoid forming these deleterious sites through direct hydrothermal synthesis or through the post-synthetic removal of adventitious Al atoms.

The zeolite faujasite (FAU) is an important structure used in 95% of the catalyst market (by mass), due to its widespread use in fluid catalytic cracking units. The synthesis of Al-free metal-substituted FAU (M-FAU) materials have not been reported, despite the widespread use of this framework in the oil and gas industries. Current methods for the synthesis of M-FAU either rely on the post-synthetic modification of Al-FAU or direct hydrothermal synthesis in the presence of AlNaO₂. For example, Trejda et al. synthesized Nb- and Ta-substituted FAU hydrothermally in the presence of sodium aluminate (needed to crystalize the structure) yet, this approach produced in M-Al-FAU materials with a Si:Al ratio equal to 2.3 (Catal. Today, 2010, 158, 170). Methods for the post-synthetic modification of Al-FAU rely on steaming at high temperatures or acid treatments to partially remove Al from framework positions and create vacant silanol nest ((SiOH)₄) defects that can be substituted with the desired metal atoms. In all previous reports, a significant amount of Al (Si:Al ratios=19-150) remains, which is detrimental for a number of relevant reactions and leads to significant reductions in yields and selectivities. Consequently, the advent of synthetic methods to produce Al-free M-FAU would be impactful for numerous industrially practiced catalytic reactions including alkene epoxidation and biomass upgrading.

The problem is millions of metric tons of zeolites are synthesized annually for production of chemicals; yet, there is no process for creating Al-free FAU for desired applications. Accordingly, there is a need for a process that allows these inexpensively obtained zeolites to be modified to perform new catalytic reactions that are relevant for more efficient production of chemicals and fuels from fossil resources or renewable biomass resources and as selective adsorbent materials.

### SUMMARY

This disclosure shows that sequential treatments of Al-FAU in concentrated HNO₃ removes nearly all of the Al within these materials and increase Si:Al ratios from 15 to values greater than 900. The incorporation of base and early-transition metals into the FAU framework preserves the crystallinity of these materials and is confirmed using a combination of techniques including X-ray diffraction, N₂ volumetric adsorption, diffuse-reflectance UV-vis spectroscopy, ²⁹Si nuclear magnetic resonance spectroscopy, and infrared spectroscopy. Infrared spectra of pyridine adsorbed within M-FAU synthesize by these methods show the presence of Lewis acid sites and undetectable numbers of Brønsted acid sites, within the sensitivity of the methods used.

Accordingly, this disclosure provides a modified zeolite comprising dealuminated faujasite that has a crystalline framework and micropores, wherein a metal heteroatom (M) is integrated into a dealuminated node of the dealuminated faujasite via a M-O—Si linkage;

wherein the modified zeolite has a silicon to aluminum (Si:Al) mole ratio of about 200 or greater and a silicon to integrated metal (Si:M) mole ratio of about 15 or greater.

In various embodiments, the nodes comprise one or more integrated metals (M) other than aluminum, such as titanium (see FIG. 1a). In various embodiments, the integrated metals are exposed at the surface of the micropores (see FIG. 1b).

This disclosure also provides a method for forming the modified zeolite as described above comprising:
(a) contacting unmodified faujasite (FAU) and a mineral acid at a reflux temperature to form a dealuminated faujasite comprising dealuminated nodes;
(b) filtering, rinsing and drying the dealuminated faujasite; and
(c) repeating steps a) and b) optionally one or more times;
   wherein the modified zeolite is thereby formed.

Additionally, this disclosure provides a method for catalyzing an oxidation reaction comprising:

contacting the modified zeolite catalyst as described above, an oxidizing agent and a substrate under suitable catalytic reaction conditions;

wherein the substrate and oxidizing agent have a sufficiently appropriate size to enter a micropore of the modified zeolite for catalysis, wherein the substrate undergoes an oxidation reaction at an integrated metal heteroatom inside the micropore that is accessible for catalyzing the oxidation reaction.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. (a) Zeolite frameworks represented by three-letter codes showing the process of dealumination and heteroatom incorporation. (b) A representative example of an olefin oxidation occurring inside the micropores of the disclosed heteroatom substituted zeolite. (c) X-ray diffractograms for Al-, Si-, Ti-, Nb-, Ta-, and Sn-FAU. Diffractograms are vertically offset for clarity.

FIG. 2. (a) Nitrogen adsorption isotherms (77 K; offset by 100 cm³ g⁻¹ for clarity) and (b) pore-size distributions for Al-FAU, Si-FAU, and Ti-FAU. The pore-size distributions were calculated using a cylindrical pore model with NLDFT. Pore-size distributions are normalized to the 1.15 nm feature and are offset for clarity.

FIG. 3. Tauc plots for Ti-, Nb-, Ta-, and Sn-FAU. Note that F(R) corresponds to the Kubelka-Munk pseudo-absorbance. All spectra were normalized to the most-intense feature and are offset for clarity. Optical band gaps were calculated from regressing the linear portion of the leading edge to a value of zero.

FIG. 4. ²⁹Si direct polarization MAS-NMR spectra of Al-, Si-, Ti-, Nb-, Ta-, and Sn-FAU. Spectra are normalized to the Q⁴ feature and scaled to the indicated value (e.g., ×5 magnification for Si-FAU). The intense Q⁴ feature has been truncated and spectra are vertically offset for clarity. FIG. 14 shows ²⁹Si MAS-NMR spectra that includes the Q⁴ feature.

FIG. 5. Infrared spectra of dehydrated Al-, Si-, Ti-, Nb-, Ta-, and Sn-FAU in flowing He (50 cm³ min⁻¹, 573 K). Spectra have been normalized to ν(Si—O—Si) at 1865 cm⁻¹ and are vertically offset for clarity.

FIG. 6. Infrared spectra of Al-FAU, Ti-FAU, Nb-FAU, Ta-FAU, and Sn-FAU in contact with gaseous pyridine (0.25 kPa, 101 kPa He, 473 K). All spectra were normalized to the most-intense feature between 1700-1400 cm⁻¹ and are offset for clarity. Numbered lanes correspond to expected regions for vibrational modes of pyridine coordinated to Lewis acid sites (LA; 4), Brønsted acid sites (BA; 2), and both Brønsted and Lewis acid sites (BA+LA; 1 and 3).

FIG. 7. (a) Turnover rates for C₈H₈ epoxidation (bars) and H₂O₂ selectivities towards epoxidation products (black o) over Ti-BEA, Ti-FAU, and Ti—SiO₂ with the characteristic pore dimension indicated. All reactions were run at standard conditions that lead to rates that are proportional to C₈H₈ concentrations (0.01 M C₈H₈, 0.01 M H₂O₂ in CH₃CN, 313 K). (b) Proposed series of elementary steps for the epoxidation of C₈H₈ over Ti-based catalysts. The symbol [symbol] represents a quasi-equilibrated step, while [symbol] represents a kinetically relevant step.

FIG. 8. Turnover rates for C₈H₈ epoxidation as a function of (a) [C₈H₈] (0.01 M H₂O₂) and (b) [H₂O₂] (3-10⁻³ M C₈H₈) on Ti-FAU (▪), Ti-BEA (▴), and Ti—SiO₂ (▾) in CH₃CN at 313 K. Dashed lines represent fits to equation 4. Data for Ti-BEA and Ti—SiO₂ are adapted from ref 23. Errors in C₈H₈O formation rates are <10% and error bars are omitted for clarity.

FIG. 9. Transition state equilibrium constants for the formation of Ti—OOH—C₈H₈^{‡} as a function of inverse temperature over Ti-FAU (i), Ti-BEA (A), and Ti—SiO₂ (V) under conditions that result in Ti—OOH MARI (3-10⁻³ M C₈H₈, 0.01 M H₂O₂ in CH₃CN). Dashed lines represent fits to equation 8 (i.e., the Eyring equation), whose slopes and intercepts are proportional to ΔH_{App}^{‡} and ΔS_{App}^{‡}, respectively.

FIG. 10. X-ray diffractograms of (a) Ti-BEA and (b) TS-1 indicating that these materials contain diffraction features characteristic of the BEA and MFI zeolite frameworks, respectively.

FIG. 11. Diffuse Reflectance UV-vis of Ti-BEA, Ti—SiO₂, and TS-1. Tauc plots for Ti-BEA, Ti—SiO₂, and TS-1. Note that F(R) corresponds to the Kubelka-Munk pseudo-absorbance. All spectra were normalized to the most-intense feature and are vertically offset for clarity.

FIG. 12. X-ray diffraction to show changes in lattice spacing for Al-, Si-, and Ti-FAU. Diffractograms are vertically offset for clarity.

FIG. 13. Nitrogen volumetric adsorption isotherms (77 K) for Al-, Si-, and Ti-FAU. Adsorption isotherm types are offset for clarity.

FIG. 14. (a)²⁹Si direct polarization MAS-NMR spectra of Al-, Si-, Ti-, Nb-, Ta-, and Sn-FAU. Panel (b) shows changes in the full width-half max (FWHM) of the Q⁴ features within each M-FAU. All spectra are normalized to the Q⁴ feature and are offset for clarity.

FIG. 15. Example peak fitting procedure for ²⁹Si MAS-NMR spectra (▪) of Si-FAU. The red curve represents NMR features attributed to Q⁴ sites, while the blue curve represents those belonging to Q³ sites; the black curve is the cumulative fit. In all cases, Lorentzian curves were used for the fitting procedure. Values of ϕ_{NMR} were estimated by dividing the area under the blue curve to that of the black curve.

FIG. 16. Infrared spectra (▪) of dehydrated Si-FAU (573 K, He). Gaussian curves were used to fit the data in all cases. The green curve represents ν(O—H) of isolated SiOH, the blue curves are ν(O—H) resulting from hydrogen-bonded SiOH (e.g., (SiOH)₄), and red curves represent ν(Si—O—Si) overtone stretches. The black curves represent cumulative fits.

Six gaussian curves were chosen to fit the infrared spectra of M-FAU in order to capture the proper curvature and yield a R² value >0.995. Fitting procedures using three-to-five gaussian curves yielded nearly identical values of $m.

FIG. 17. Turnover numbers as a function of time for the epoxidation of 2,4-dimethylstyrene (0.1 M 2,4-dimethylstyrene, 0.1 M H₂O₂, in CH₃CN, 313 K) over Ti-FAU (▪), Ti-BEA (▴), Ti—SiO₂ (▾), and TS-1 (♦). Dashed curves are intended to guide the eye. The plot shows the turnover numbers for 2,4-dimethylstyrene epoxidation over Ti-FAU, Ti-BEA, Ti—SiO₂, and TS-1. Turnover numbers (and rates) for 2,4-dimethylstyrene epoxidation are greatest within Ti-FAU and decrease in the order Ti-BEA, Ti—SiO₂, and TS-1. Notably, rates of 2,4-dimethylstyrene epoxidation are immeasurable on TS-1 because TS-1 possesses pores that are 0.55 nm in diameter, which is smaller than the kinetic diameter of 2,4-dimethylstyrene (e.g., m-xylene has a kinetic diameter of 0.68 nm) (M. Jahandar Lashaki, et al, J. Hazard. Mater., 2012, 241-242, 154-163), which precludes the diffusion and reaction of 2,4-dimethylstyrene within the MFI framework. The increased turnover numbers for 2,4-dimethylstyrene oxide formation within Ti-FAU relates to the increased entropic freedom of the transition state for epoxidation within Ti-FAU compared to Ti-BEA, with the increased enthalpic stabilization relative to Ti—SiO₂.

### DETAILED DESCRIPTION

Heteroatom framework-substituted zeolites are important materials that enable shape- and size-selective catalysis. The efficacy of these materials for desired catalytic reactions depends critically on dispersive interactions between the microporous void of the zeolite and the reactant molecules stabilized within it.

Synthesis of zeolites that contain aluminum is well-developed and relatively cost effective, however, the synthesis of these same structures when they contain other catalytically active elements (e.g., titanium, tin, niobium, tantalum, hafnium, zirconium, tungsten, molybdenum, etc.) is more time consuming, less reliable, provide lower yields, gives lower densities of active metal atoms per gram catalyst, requires expensive synthesis additives (called structure directing agents), or the methods simply do not exist. A pre-existing example is the synthesis of two widely used zeolite catalysts-titanium beta (Ti-BEA) and tin beta (Sn-BEA). These catalysts can be created directly by hydrothermal methods, however, the dealumination of a standard aluminum beta (Al-BEA) by extended thermal treatments in concentrated acids (e.g., nitric, sulfuric, or hydrochloric acids) can remove the aluminum atoms leaving behind point defects. The adsorption of Ti or Sn metal complexes onto these point defects from a liquid solvent (e.g., organic phases) followed by drying and oxidation at elevated temperatures (>400 C) integrates these metal atoms into the crystal structure in a catalytically active form.

Here, we develop a post-synthetic method to synthesize base and transition metal-substituted (Ti, Nb, Ta, and Sn) FAU with ultralow Al contents (Si:Al >900), which is confirmed using X-ray diffraction, elemental analysis, and N₂ volumetric adsorption and ²⁹Si MAS-NMR, DRUV-vis, and IR spectroscopic characterization. Ti-FAU selectively stabilized styrene (C₈H₈) within its pores during epoxidation with H₂O₂. Turnover rates for C₈H₈ epoxidation within Ti-FAU are 2- and 7-fold greater than in analogous Ti-BEA and Ti—SiO₂, respectively; yet, turnover rates of H₂O₂ decomposition are similar for all three materials. Consequently, Ti-FAU gives greater rates and selectivities for this reaction than common Ti-bearing silicates. The mechanism for epoxidation remains constant for all Ti-silicates examined (i.e., Ti-FAU, Ti-BEA, and Ti—SiO₂). Therefore, the improved performance of Ti-FAU reflects differences in activation free energies for epoxidation that show an enthalpic preference in Ti-FAU relative to Ti—SiO₂ and an entropic gain relative to Ti-BEA. These results demonstrate the synthesis of M-FAU with ultralow Al contents are useful for catalytic reactions involving bulky reactants that cannot occur in smaller pore zeotype materials (Ti-MFI), that exhibit deactivation due to changes in Ti-atom coordination (e.g., Ti—SiO₂), and that are prone to losses catalyzed by residual Brønsted acid sites (e.g., epoxidations, oxidations, and isomerization reactions).

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture.

An “effective amount” refers to an amount effective to bring about a recited effect, such as an amount necessary to form products in a reaction mixture. Determination of an effective amount is typically within the capacity of persons skilled in the art, especially in light of the detailed disclosure provided herein. The term “effective amount” is intended to include an amount of a compound or reagent described herein, or an amount of a combination of compounds or reagents described herein, e.g., that is effective to form products in a reaction mixture. Thus, an “effective amount” generally means an amount that provides the desired effect.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983).

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

As used herein, the term “substituted” or “substituent” is intended to indicate that one or more (for example, 1-20 in various embodiments, 1-10 in other embodiments, 1, 2, 3, 4, or 5; in some embodiments 1, 2, or 3; and in other embodiments 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound.

A “solvent” as described herein can include water or an organic solvent. Examples of organic solvents include hydrocarbons such as toluene, xylene, hexane, and heptane; chlorinated solvents such as methylene chloride, chloroform, and dichloroethane; ethers such as diethyl ether, tetrahydrofuran, and dibutyl ether; ketones such as acetone and 2-butanone; esters such as ethyl acetate and butyl acetate; nitriles such as acetonitrile; alcohols such as methanol, ethanol, and tert-butanol; and aprotic polar solvents such as N,N-dimethylformamide (DMF), N,N-dimethylacetamide (DMA), and dimethyl sulfoxide (DMSO). Solvents may be used alone or two or more of them may be mixed for use to provide a “solvent system”.

The phrase “one or more” is readily understood by one of skill in the art, particularly when read in context of its usage. For example, one or more metals means each of the one or more metals can be different.

Zeolites are microporous, aluminosilicate minerals that can be used as catalysts for chemical reactions. Faujasite is an example of a zeolite. The faujasite framework consists of sodalite cages which are connected through hexagonal prisms. The pore, which is formed by a 12-membered ring, has a relatively large diameter of 7.4 Å. The supercage comprises an inner cavity of the has a diameter of 12 Å and is surrounded by 10 sodalite cages.

The term “node” refers to a point at which lines or pathways intersect or branch; e.g., a central or connecting point. As used herein, the node refers to the point where a metal atom may or may not be positioned within the crystalline framework of the zeolite. The node may comprise an aluminum or titanium atom or another metal atom as disclosed herein. The node may also comprise a void in place of, for example, an aluminum atom that was positioned at the node, thereby filling the void.

Early transition metals are defined as metals on the left side of the periodic table from group 3 to group 7. Late transition metals are defined as metals on the right side of the d-block, from group 8 to 11. Other metals can be selected from group 12-14.

### EMBODIMENTS OF THE INVENTION

This disclosure provides a modified zeolite comprising dealuminated faujasite (FAU) that has a crystalline framework and micropores, wherein a metal heteroatom (M) is integrated into a dealuminated node of the dealuminated faujasite via a M-O—Si linkage;

wherein the modified zeolite has a silicon to aluminum (Si:Al) mole ratio of about 200 or greater and a silicon to integrated metal (Si:M) mole ratio of about 15 or greater.

In additional embodiments, the micropores have diameters of about 1 nm to about 2 nm. In other embodiments, the diameters are about 0.4 nm, about 0.6 nm, about 0.8 nm, about 1.5 nm, about 2.5 nm, or about 3 nm.

In further embodiments, the mole ratio of silicon to aluminum is greater than 200. In other embodiments, the ratio is about 10, about 15, about 20, about 25, about 50, about 100, about 150, about 200, about 250, about 300, about 350, about 400, about 450, about 500, about 550, about 600, about 650, about 700, about 750, about 800, about 850, about 900, about 950 or more, about 1000, about 1100, about 1200, about 1300, about 1400, about 1500, about 2000, about 3000, or about 4000.

In some other embodiments, the mole ratio of silicon to aluminum is about 500 to about 2000, about 750 to about 1000, about 900 to about 1500, or about 950 to about 2000. It is noted that sufficiently high Si:Al ratio is required to reduce or eliminate deleterious side products from reactions that occur at Brønsted acid sites associated with the Al atoms. For example, when performing epoxidations diol formation become problematic when the Si:Al ratio is not sufficiently high.

In various embodiments, the modified zeolite is dealuminated: mordenite (MOR), chabazite (CHA), Socony Mobil-five (MFI), or Mobil Composition of Matter-twenty-two (MWW). It is noted that zeolites are designated by their three-letter codes, as known to persons of ordinary skill in the art.

In additional embodiments, the nodes comprise one or more integrated metals (M), early transition metals, late transition metals, or other metals/metaloids. In other embodiments, the early transition metals, late transition metals, lanthanides, or other metals are exposed at the inner surface of the micropores.

In additional embodiments, the early transition metals are one or more of titanium, tin, niobium, iron, cerium, lanthanum, tantalum, zirconium, hafnium, molybdenum, and tungsten. In other embodiments, the other metals are tin and/or germanium.

In further embodiments, the early transition metals are integrated as M-O—Si linkages, or the early transition metals are bonded to siliceous nests as M-O—Si linkages, as can be determined by diffuse reflectance UV-VIS spectroscopy, XRD, and FTIR spectroscopy. In some other embodiments, the early transition metals are essentially free of M-O-M linkages as can be determined by diffuse reflectance UV-VIS and Raman spectroscopy.

In yet other embodiments, the zeolite is essentially free of silanol as can be determined by infrared spectroscopy. In some embodiments, the (SiOH)₄ (formed by dealumination) is consumed when a metal (other than aluminum) is nested or bonded to the silanol moieties. In some preferred embodiments, the dealuminated zeolite is FAU, the integrated metal is titanium, and the mole ratio of silicon to aluminum is about 200 to about 2000.

This disclosure also provides a modified faujasite zeolite comprising a crystalline framework, micropores, and nodes in the crystalline framework comprising integrated titanium, wherein the mole ratio of silicon to aluminum in the modified faujasite is 900 or more and the modified faujasite comprises less aluminum compared to an unmodified faujasite zeolite.

This disclosure additionally provides a modified faujasite zeolite comprising a dealuminated crystalline framework, micropores, and nodes in the crystalline framework void of aluminum, wherein the mole ratio of silicon to aluminum in the modified faujasite is 900 or more and the modified faujasite comprises less aluminum compared to an unmodified faujasite zeolite.

Also, this disclosure provides a method for forming a modified zeolite comprising:
(a) contacting a parent zeolite and a mineral or organic acid one or more times to form a dealuminated zeolite wherein the dealuminated zeolite comprises a crystalline framework, micropores, and nodes in the crystalline framework void of aluminum;
(b) filtering and rinsing the dealuminated zeolite one or more times; and
(c) heating the dealuminated zeolite at a first temperature of greater than 400° C. one or more times;
   wherein the formed modified zeolite has a mole ratio of silicon to aluminum of greater than 150.

In some embodiments, the contacting is at the reflux temperature (or lower) of the mineral acid or organic acid solutions. In other embodiments, the mineral acid is nitric acid, sulfuric acid, hydrochloric acid, hydrobromic acid, hydroiodic acid, or a combination thereof. In yet other embodiments, the organic acid is acetic acid, formic acid, oxalic acid, citric acid, ethylenediaminetetraacetic acid, other chelating organic acid complexes, or a combination thereof. In various embodiments, the micropores have diameters of about 1 nm to about 2 nm, or about 0.5 nm to about 3 nm.

In other embodiments, the method comprises integrating heteroatoms into the nodes of the dealuminated zeolite. In additional embodiments, the modified zeolite is formed at a second temperature of greater than 400° C. In some embodiments the first temperature or second temperature is independently about 200° C. to about 400° C., about 400° C. to about 600° C., about 600° C. to about 800° C., about 450° C., about 500° C., or about 550° C.

In other embodiments, the method further comprises integrating a metal heteroatom into a dealuminated node of the dealuminated faujasite. In various embodiments, the heteroatoms are integrated by anhydrous liquid-phase grafting. In additional embodiments, the integrated heteroatoms are early transition metals, late transition metals, or other metals. In other various embodiments, the early transition metals, late transition metals, or other metals are exposed at the surface of the micropores. In further embodiments, the parent zeolite is faujasite (FAU), the early transition metals are titanium, and the mole ratio of silicon to aluminum is about 200 to about 2000.

Additionally, this disclosure provides a method for catalyzing a chemical reaction comprising contacting a molecule and a zeolite catalyst under suitable catalytic reaction conditions,

wherein the zeolite catalyst comprises a dealuminated zeolite having a crystalline framework, micropores, and nodes in the crystalline framework integrated with one or more integrated metals, and the zeolite catalyst has a mole ratio of silicon to aluminum of greater than 150; and

the molecule has a sufficiently small size to enter the micropores and undergo the catalytic chemical reaction.

In some other embodiments, the early transition metals, late transition metals, or other metals are exposed at the surface of the micropores. In yet other embodiments, the micropores have diameters of about 1 nm to about 2 nm. In further embodiments, the contacting comprises an oxidizing agent, the catalyzed chemical reaction affords an oxidized molecule, and the turnover rate of the catalyzed chemical reaction is at least two-times greater than a corresponding zeolite catalyst that has a mole ratio of silicon to aluminum of less than 150 or is not dealuminated FAU.

In additional embodiments, the molecule is an olefin, the oxidizing agent is a peroxide, the crystalline framework of the zeolite catalyst is FAU, the early transition metal is titanium, and the mole ratio is about 200 to about 2000. In some embodiments, oxidizing agent is a peroxide or hydroperoxide.

In yet other embodiments, the suitable catalytic reaction conditions comprise the contacting at the early transition metals exposed at the surface of the micropores, a polar aprotic solvent, and a catalytic reaction temperature of about −10° C. to about 80° C. In some embodiments the solvent is water or a polar protic solvent. In other embodiments, the reaction temperature is about 20° C. to about 60° C., about 25° C. to about 55° C., about 55° C. to about 155° C., or about 100° C. to about 200° C.

Heteroatom Substituted Zeolite FAU with Ultralow Al Contents for Liquid-Phase Oxidation Catalysis. Comparisons of the rates, selectivities, and yields for styrene (C₈H₈) epoxidation with H₂O₂ over Ti-FAU to that in other Ti-silicates demonstrates advantages of locating Ti active sites within the supercages of the FAU structure. Turnover rates for styrene epoxidation over Ti-FAU are 2- and 7-fold greater than those in Al-free Ti-BEA (0.65 nm pore diameter) and Ti—SiO₂ (5.4 nm pore diameter), respectively. Rates of H₂O₂ decomposition (2.7±0.9 (mmol H₂O₂)(mol Ti·s)⁻¹) remain constant for all structures, and therefore, do not depend on the characteristic dimensions of these Ti-silicate catalysts. Consequently, Ti-FAU gives greater selectivities and greater turnover rates for styrene epoxidation than Ti-BEA and Ti—SiO₂. Mechanistic interpretation of epoxidation rates measured as a function of reactant concentrations show that all Ti-silicates irreversibly activate H₂O₂ to form Ti—OOH intermediates that react with C₈H₈ in a kinetically relevant step to form styrene oxide. Measured activation enthalpies for C₈H₈ epoxidation with pore size among these materials (e.g., Ti—SiO₂ (37±4 kJ mol⁻¹)>Ti-FAU (22±2 kJ mol⁻¹) >Ti-BEA (9±2 kJ mol⁻¹)), which shows that the micropores of BEA enthalpically stabilize C₈H₈ epoxidation transition states relative to the Ti—OOH reactive intermediate. Apparent activation entropies, however, become increasingly negative with decreasing pore size (−155 to −234 J mol⁻¹ K⁻¹) suggesting that the dispersive interactions between C₈H₈ epoxidation transition states and the pore walls of BEA results in the significant loss of translational and vibrational entropy. Consequently, the greater rates and selectivities for C₈H₈ epoxidation within Ti-FAU arise from the balance of enthalpy-entropy compensation effects that depend on the pore structure of the silicate. The 1.2 nm voids of FAU give enthalpic benefits for C₈H₈ epoxidation, relative to Ti—SiO₂, yet does not incur the same entropic losses suffered within Ti-BEA. Collectively, these methods and data provide a pathway to synthesize Al-free M-FAU materials, from synthetic or natural Al-FAU, that can be used for adsorption, separations, or catalysis, and which can allow for the access of other Ti-zeolite structures through inter-zeolitic transformations.

### Results and Discussion

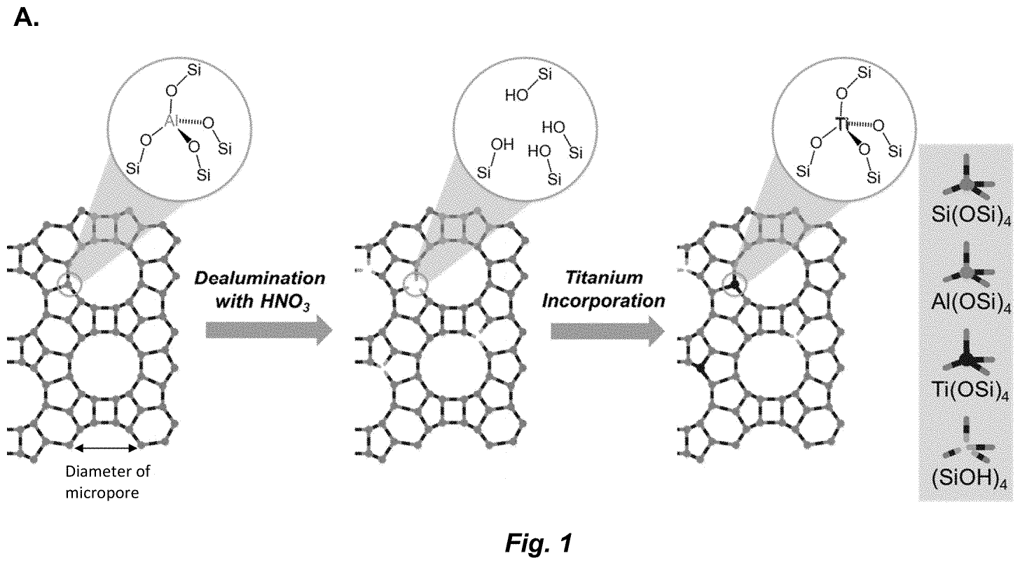

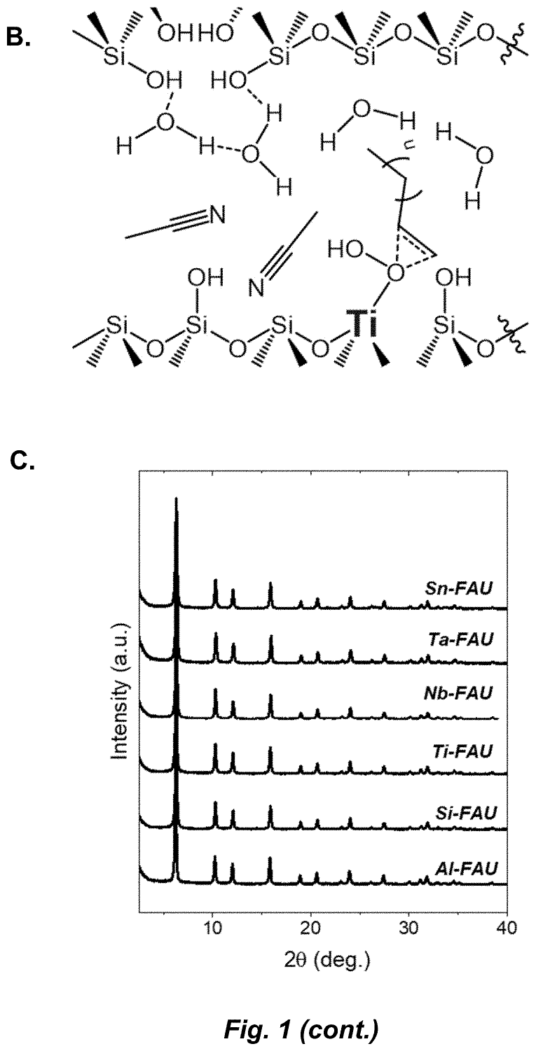

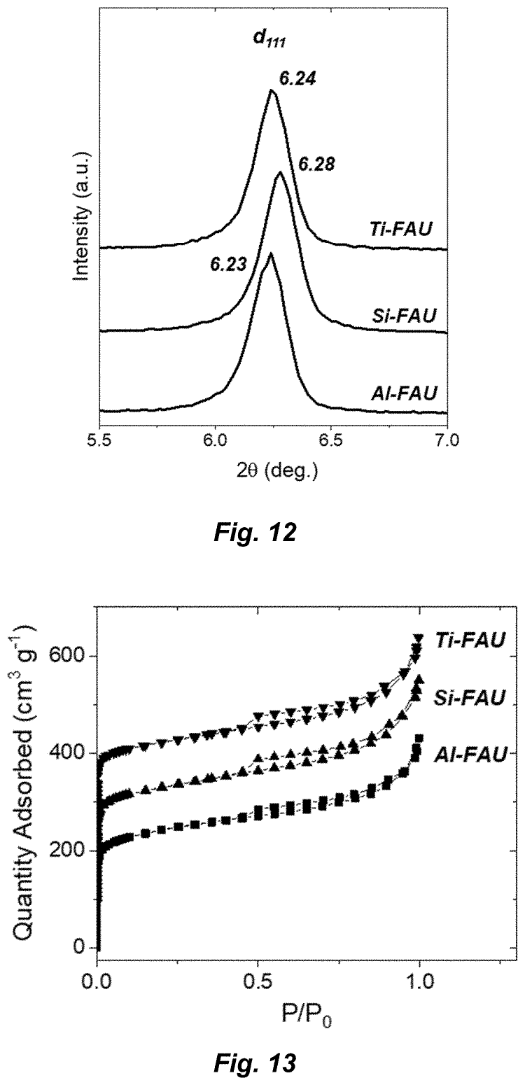

Crystallinity of FAU is Maintained During Post-Synthetic Modification. Titanium, niobium, tantalum, and tin framework-substituted FAU (M-FAU) were synthesized through the post-synthetic modification of H⁺-form Al-FAU. FIG. 1c shows X-ray diffractograms for M-FAU all possess features indicative of the FAU framework. The relative crystallinity for each M-FAU was estimated by taking the ratio of the 10.3° and 6.3° and assuming 100% crystallinity within Al-FAU. There is no apparent loss in crystallinity upon post-synthetic modification (Table 1), which suggests that dealumination and subsequent metal substitution does not alter the FAU framework. The din spacing shifts from 6.23° to 6.28° (FIG. 12) upon dealumination, which is consistent with the contraction of the FAU framework resulting from appreciable densities of silanol nests ((SiOH)₄). The incorporation of heteroatoms shifts the din spacing to 6.24° (in the case of Ti), which indicates that the M atoms are integrated into the (SiOH)₄ nests formed by dealumination to produce framework heteroatoms sites with the capacity for catalysis.

TABLE 1
Si:Al and Si:M Ratios, Metal Loadings, Relative Crystallinities, Optical
Band Gaps, BET Surface Areas, Fraction of Si Atoms Existing as SiOH (ϕ_{NMR}),
and Relative Densities of Hydrogen-Bonded SiOH (φ_{IR}) within M—FAU.

| | | | Metal | | Band | | | |
|---|---|---|---|---|---|---|---|---|
| | | | Loading | Crystallinity | Gap | BET Surface | | |
| Sample | Si:Al^{a} | Si:M^{a} | (wt. %)^{a} | (%)^{b} | (eV)^{c} | Area (m² g⁻¹)^{d} | ϕ_{NMR}^{e} | φ_{IRf} |
| Al—FAU | 14.8 | 14.8 | 2.85 | 100 | — | 800 | —^{g} | —^{h} |
| Si—FAU | >900 | — | — | 96 | — | 760 | 0.091 | 3.4 ± 0.2 |
| Ti—FAU | >900 | 39.1 | 1.97 | 106 | 4.4 | 740 | 0.051 | 2.1 ± 0.1 |
| Nb—FAU | >900 | 37.9 | 3.85 | 105 | 4.8 | — | 0.049 | 2.5 ± 0.2 |
| Ta—FAU | >900 | 47.3 | 5.90 | 108 | 4.8 | — | 0.055 | 2.7 ± 0.2 |
| Sn—FAU | >900 | 42.3 | 4.42 | 99 | 4.2 | — | 0.053 | 1.9 ± 0.1 |

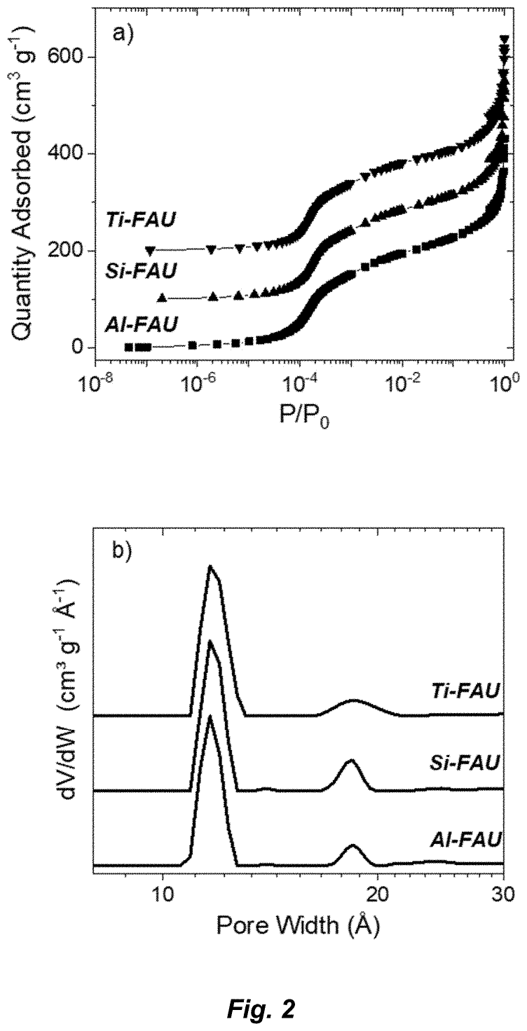

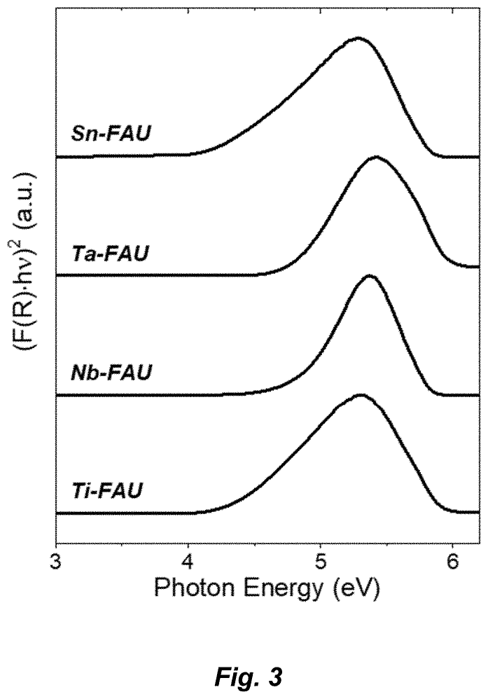

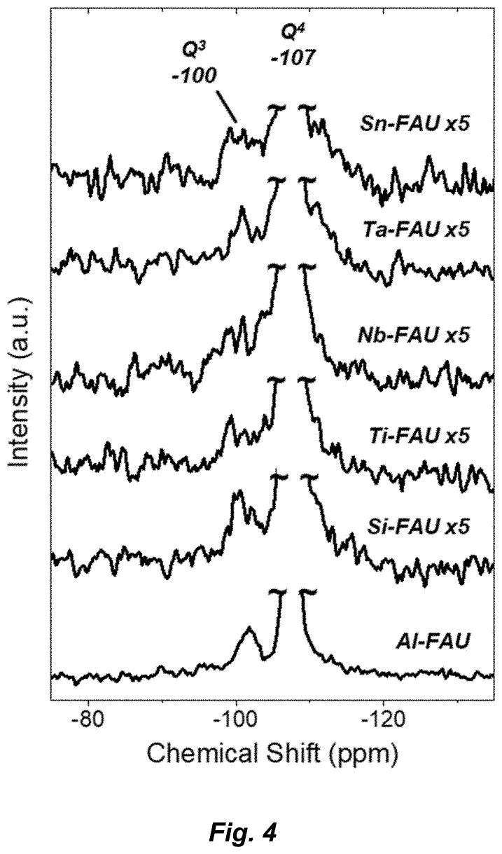

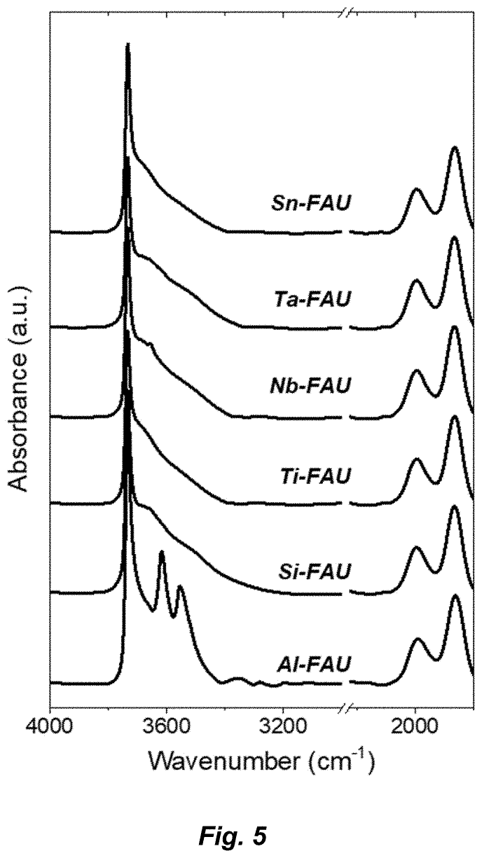

^{a}Measured by EDXRF.
^{b}Determined by DRUV-vis spectroscopy by extrapolating the linear portion of the leading edge of the corresponding Tauc plot (FIG. 3).
^{c}Estimated by taking the ratio of the intensity for the 10.3° to the 6.3° diffraction features and assuming perfect 100% crystallinity for Al—FAU.
^{d}Calculated using N₂ adsorption isotherms (FIG. 2a).
^{e}Quantified using ²⁹Si MAS-NMR (FIG. 4).
_{f}Determined from FTIR spectra of dehydrated M—FAU (FIG. 5a)
^{g}NMR features for Si atoms residing as Si(OAl)(OSi)₃ and Si(OSi)₃OH overlap and precludes determination.
^{h}ν(O—H) of H⁺ bound to Si—O—Al moieties overlap with ν(O—H) of hydrogen-bonded SiOH, which prevents quantification

Post-Synthetic Modification Does Not Form Mesopores. Nitrogen adsorption isotherms (77 K; FIG. 2a) were measured on Al-, Si-, and Ti-FAU to probe changes in the physical properties (i.e., Brunauer-Emmett-Teller (BET) surface area, pore-size distribution) of the FAU framework upon dealumination and subsequent Ti-atom incorporation. The adsorption profile for N₂ is characteristic of weak adsorbate-adsorbent interactions (Type III) and approaches micropore filling below a relative pressure (P/Po) of 10⁻⁴. As P/Po is increased, N₂ adsorption resembles a typical Type I isotherm that is characteristic of adsorption within a microporous solid. FIG. 2a contains a log-scale for the abscissa that gives the illusion that N₂ adsorption resembles a Type IV isotherm; however, the linear-scaling of P/Po (FIG. 13) reveals the Type 1 nature of N₂ adsorption.

The surface area of M-FAU was estimated using BET theory and yielded equivalent values for the three samples tested (740-800 m² g⁻¹, Table 1), which further suggests that post-synthetic modification of Al-FAU does not modify the long-range order of the framework or create sufficiently large defects that change the internal surface area. FIG. 2b shows the pore-size distributions calculated for Al-, Si-, and Ti-FAU all possess a significant feature with a characteristic pore width of ˜1.2 nm, which corresponds to the supercage within the FAU framework. The small feature around 1.8 nm may result from defects that partially connect two adjacent cages. Importantly, the similarities between the pore-size distributions, the BET surface areas, and the characteristics of N₂ adsorption all suggest that post-synthetic modification does not significantly alter the FAU framework. Nitrogen possesses a significant quadrupolar moment that may lead to specific interactions with adsorption sites, which typically prompts the use of argon as an adsorptive. The similarities between the adsorption isotherms here, however, further suggests the FAU framework is unchanged upon post-synthetic treatment.

Metal Sites Within FAU Framework are Highly Disperse. Optical band gaps reports on the speciation (and dispersion) of semi-conducting and insulating solids. FIG. 3 shows Tauc plots for M-FAU all possess a single prominent absorbance feature between 5.1 and 5.3 eV, which corresponds to the ligand-to-metal charge transfer between the 0 atoms within the framework of FAU and the M atom within the sample (e.g., charge transfer between the 2p orbitals of oxygen to the 3d orbitals of Ti⁴⁺). Table 1 shows that the band gaps for these Ti-, Nb-, Ta-, and Sn-FAU are all significantly larger those of the bulk MOₓ analogues (bulk anatase TiO₂, Nb₂O₅, Ta₂O₅, and SnO₂ have band gaps of 3.2, 3.4, 3.9, and 3.6 eV, respectively). Moreover, the band gaps for these M-FAU samples are comparable to those for hydrothermally-synthesized M-BEA and M-MFI zeolites, which suggests these metal atoms are incorporated into the FAU framework. The large band gaps for the M-FAU within this study, relative to bulk MOₓ, suggest that metal atoms are well isolated within the zeolite framework and that these samples contain negligible amounts of M-O-M linkages.

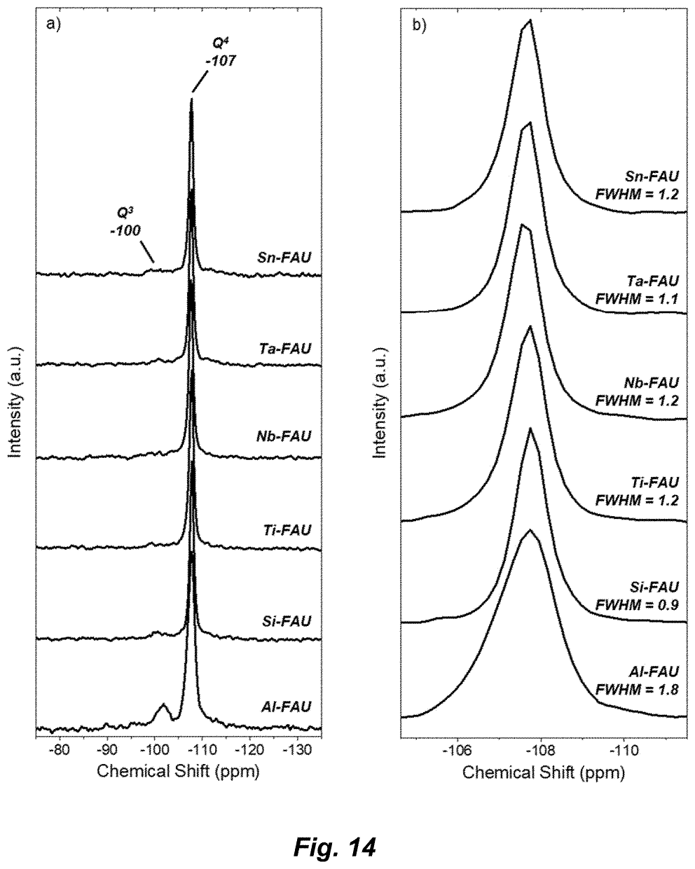

Grafting of Metal Ions Leads to Reduction in the Number of SiOH. The fraction of Si atoms that exist as SiOH within each M-FAU can provide indirect evidence for the formation and elimination of (SiOH)₄ upon dealumination and subsequent metal-ion incorporation. FIG. 4 shows ²⁹Si MAS-NMR spectra that contain distinct NMR features at chemical shifts of −107 and −100 ppm on all M-FAU. The feature at −107 ppm corresponds to Si atoms within the FAU framework coordinated to four siloxane functions (i.e., Si(OSi)₄, denoted as Q⁴ sites) or to three siloxanes and a substituted metal atom (i.e., Si(OSi)₃OM; M=Ti, Nb, Ta, Sn). FIG. 14 shows the full width-half max of the Q⁴ feature increases from 0.9 ppm for Si-FAU to 1.1-1.2 ppm for M-FAU, which indicates this feature contains contributions from both Si(OSi)₄ and Si(OSi)₃OM moieties. The broad feature at −100 ppm for Al-FAU corresponds to Si atoms that possess an adjacent Al atom (i.e., Si(OAl)(OSi)₃). For all other M-FAU, the small feature at −100 ppm corresponds to Si atoms that possess a single pendant hydroxyl (i.e., Si(OSi)₃OH, denoted as Q³ sites).

The fraction of Si atoms that reside as Si(OSi)₃OH (ϕ_{NMR}) is described by the fraction of Q₃ sites to the sum of Q³ and Q⁴ sites

$${ϕ}_{N M R} = \frac{{A}_{{Q}^{3}}}{{A}_{{Q}^{3}} + {A}_{{Q}^{4}}} \qquad ( 1 )$$

where A_{Q}_{³} and A_{Q}_{⁴} are the areas of the deconvoluted ²⁹Si MAS-NMR features for Q³ and Q⁴ sites, respectively (Section S3.1) shows representative peak fits to determine the Q³ and Q⁴ areas). Table 1 shows that #mx decreases from 0.09 to 0.05 upon framework substitution into Si-FAU samples, which follows expectations based upon the liberation of HCl or EtOH upon metal grafting onto (SiOH)₄ and formation of Si—O-M linkages.

A statistical distribution of (SiOH)₄ within the framework before and after the substitution of M atoms (assuming a final Si:M ratio of ˜40; Table 1) suggests a 35% reduction in the total number of (SiOH)₄, which qualitatively agrees with the measured changes in ϕ_{NMR} between Si-FAU and M-FAU (˜44% reduction). Values of ϕ_{NMR}, however, do not directly represent the density of (SiOH)₄ (i.e., SiOH formed upon dealumination), because these values encompass all Si atoms that exist as SiOH including those at point defects and on the external surface of FAU particles.

Metal Ions Within M-FAU Occupy (SiOH)₄ and Are Lewis Acidic. Isolated SiOH and hydrogen-bonded (SiOH)₄ moieties in M-FAU materials possess distinct ν(O—H) that can be used to yield semiquantitative estimates for the relative density of (SiOH)₄ groups. FIG. 5 shows IR spectra of dehydrated M-FAU samples (573 K in He) all possess distinct features at 1990 and 1865 cm⁻¹, which correspond to ν(Si—O—Si) overtones that reflect contributions from the FUA framework. The complex broad features between 3750-3400 cm⁻¹ correspond to ν(O—H) modes of distinct types of SiOH. The sharp feature at 3740 cm⁻¹ within all M-FAU corresponds to ν(O—H) of isolated SiOH that do not interact with nearby hydrogen bond-acceptor moieties. Within Al-FAU, the sharp features at 3640 and 3550 cm⁻¹ are characteristic of Brønsted acid sites within the supercage and sodalite cages of FAU, respectively. The broad ν(O—H) feature extending between 3750-3400 cm⁻¹ in M-FAU (M=Si, Ti, Nb, Ta, and Sn) samples corresponds to (SiOH)ₓ (e.g., (SiOH)₄) that contain adjacent hydrogen-bonded —OH groups.

Relative densities of isolated SiOH and (SiOH)₄ among M-FAU samples are estimated by normalizing each infrared spectra to the total number of framework bonds, which is assumed to be constant among these samples and is represented by the intensity of the ν(Si—O—Si) (1865 cm⁻¹) within this study. Peak fitting (Section S3.2) of the ν(O—H) region allows for the deconvolution of isolated SiOH (3740 cm⁻¹) and (SiOH)₄ groups (3300-3740 cm⁻¹); where the ratio of the cumulative area of ν(O—H) for (SiOH)₄ (A_{(SiOH)}_{₄}) normalized to that of ν(Si—O—Si) (A_{Si—O—Si}) yields a quantitative estimate for the relative density of (SiOH)₄ (ϕ_{IR}) among M-FAU materials.

$${ϕ}_{IR} = \frac{{A}_{{( S i O H )}_{4}}}{{A}_{Si—O—Si}} \qquad ( 2 )$$

Table 1 shows values of ϕ_{IR} decrease from 3.4±0.2 for Si-FAU to ˜2±0.1 for M-FAU. The decrease in ϕ_{IR} (˜40%) for Ti- and Sn-FAU is quantitatively consistent with the expected loss of (SiOH)₄ (˜35%) upon metal-atom incorporation. Nb- and Ta-FAU are pentacoordinate and possess a pendant —OH, which obviates how changes in ν(O—H) solely result in the loss of (SiOH)₄; however, the general trend in decreasing ϕ_{IR} a for these materials suggests the incorporation of Nb and Ta atoms into (SiOH)₄. Collectively, the data and interpretation from X-ray diffraction, diffuse reflectance, UV-vis, N₂ volumetric adsorption, ²⁹Si MAS-NMR, and IR spectroscopy experiments suggest that the post-synthetic modification procedure presented here first generates a nearly siliceous FAU material with a number of (SiOH)₄ nests equal to the original number of Al atoms, and second, substitutes the desired M atoms (M=Ti, Nb, Ta, Sn) into the zeolite framework.

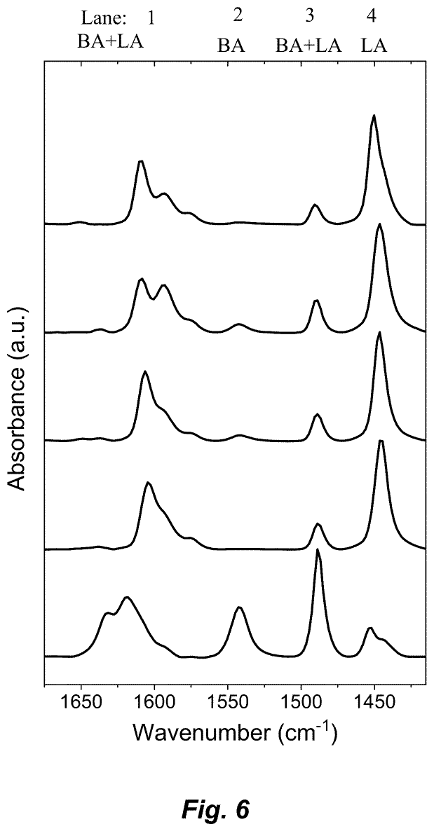

Heteroatom-substituted zeolites often act as solid Lewis acid catalysts, where the efficacy of these materials depends strongly on the electron affinity (described colloquially as the “Lewis acid strength”) of the active site. Pyridine molecules bound to Brønsted acid sites form pyridinium ions that possess vibrational modes distinct from pyridine molecules bound to Lewis acid sites, and these differences provides a means to discriminate between different types of acid sites within solid materials. FIG. 6 shows IR spectra of M-FAU materials in contact with dilute streams of vapor-phase pyridine (0.25 kPa, 101 kPa He, 473 K). All M-FAU possess significant absorbance features between 1650-1575 cm⁻¹ and at 1500 cm⁻¹, which correspond to vibrational modes of pyridine molecules adsorbed to either Brønsted or Lewis acid sites. The absorbance features around 1450 cm⁻¹ are assigned to the vibrational modes of pyridine adsorbed solely to Lewis acid sites, while the absorbance feature at 1540 cm⁻¹ is attributed to the vibrational modes of the pyridinium ion.

Al-FAU clearly possesses the greatest density of Brønsted acid sites among these materials. Within Ti-, and Sn-FAU the feature at 1540 cm⁻¹ is nearly indistinguishable from baseline, which suggests these materials do not possess spectroscopically observable densities of Brønsted acid sites. FIG. 6 shows that Nb- and Ta-FAU possess significant amounts of adsorbed pyridinium, because Nb and Ta atoms within zeolites are five coordinate and possess a pendant —OH, which may act as a Brønsted acid. Notably, the presence of Brønsted acid sites within Nb- and Ta-FAU cannot be due to residual Al atoms, as the vibrational features that discriminate these features are not present on Ti- or Sn-FAU, which were synthesized using the same batch of Si-FAU. For all M-substituted FAU, there is a significant increase in intensity for absorbance features that correspond to pyridine bound to Lewis acid sites, which is consistent with reports for these metal atoms substituted into other silicate frameworks. These Lewis acidic active sites constitute an important class of catalysts for a variety of reactions (see above), and the stabilization of intermediates critical to epoxidation catalysis within the FAU framework is demonstrated on Ti-FAU in the following section.

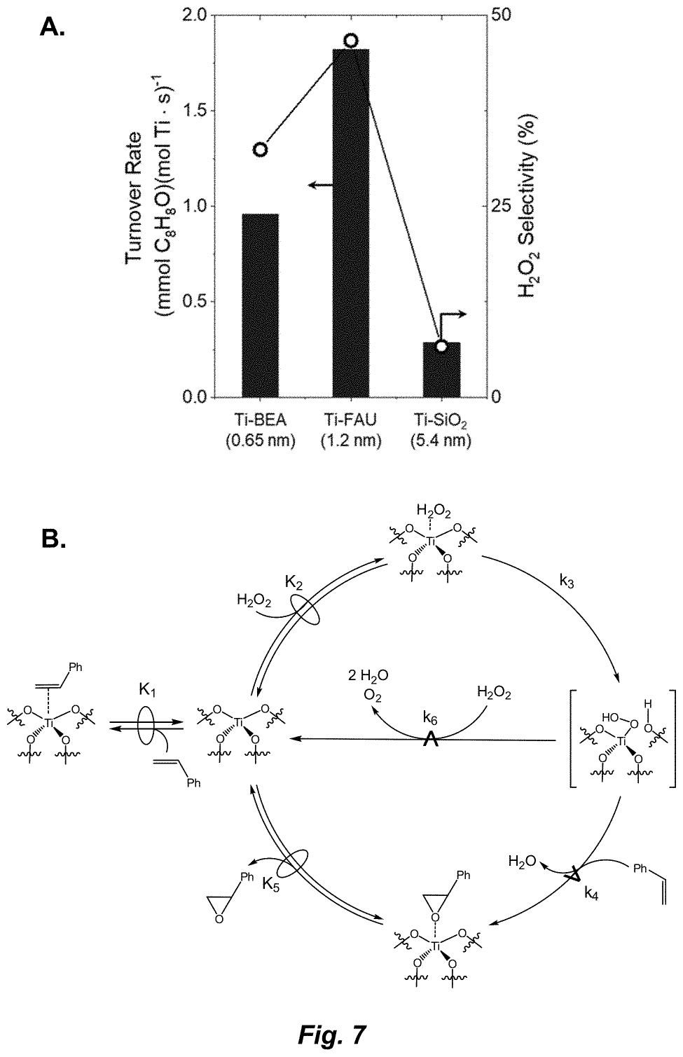

Reaction Pathways for Alkene Epoxidation with Hydrogen Peroxide. Zeolites and mesoporous silicates bind reactants by charge transfer at active sites but also permit the selective stabilization of surface intermediates through combinations of van der Waals and specific interactions among the extended zeolite surface, the solvent molecules and the reactive species. The extent of stabilization depends on both the size and shape of the confining pore and the reactive species contained within these spaces. FIG. 7a shows turnover rates for styrene (C₈H₈) epoxidation with hydrogen peroxide (H₂O₂) are significantly greater on Ti-FAU (1.2 nm supercage) than for Ti-BEA (0.65 nm pore) and Ti—SiO₂ (5.4 nm pore) at all reaction conditions examined. Specifically, epoxidation rates over Ti-FAU are greater than Ti-BEA and Ti—SiO₂ by factors of 2 and 7, respectively. H₂O₂ selectivities represent the percent of H₂O₂ molecules that are consumed by epoxidation reaction pathways and decrease in the same fashion as epoxidation turnover rates between Ti-FAU (47%), Ti-BEA (30%), and Ti—SiO₂ (6%). Rates of H₂O₂ decomposition (2.7±0.9 (mmol H₂O₂)(mol Ti·s)⁻¹) are nearly identical on Ti-FAU, Ti-BEA, and Ti—SiO₂ because the transition states for H₂O₂ decomposition are too small to experience interactions with the pore walls of the silicate hosts that differ among these materials.

The importance of ultralow Al contents is exemplified in reactions that include Al-FAU to simulate a Si:Al of 150. The presence of Al-FAU results in no measurable C₈H₈O formation; yet, rather forms 1-phenyl-1,2-ethanediol (from C₈H₈O ring opening) and 1-phenylethanol (from C₈H₈ hydration over H⁺ sites). Therefore, the differences in C₈H₈ epoxidation catalysis and the corresponding H₂O₂ selectivity must relate to how the stability of C₈H₈-derived intermediates depend on the chemical characteristics of the Ti-silicate catalyst.

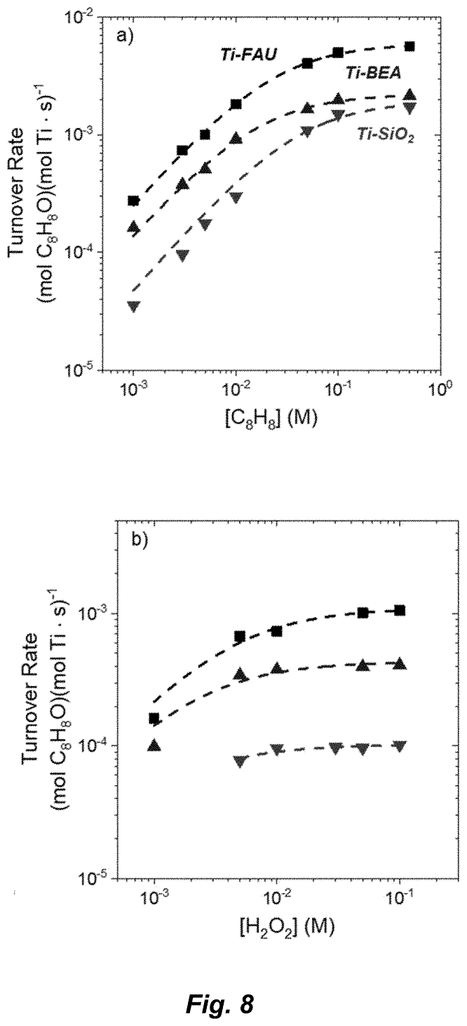

Turnover rates for (C₈H₈) epoxidation with hydrogen peroxide (H₂O₂) were measured as a function of C₈H₈ and H₂O₂ concentration to provide insight as to the mechanism for alkene epoxidation and reconcile the differences in rates and selectivities for C₈. Notably, the Ti-FAU sample used within these kinetic measurements was synthesized to contain 0.3%, by weight, Ti atoms to avoid artifacts that may arise from internal concentration gradients (i.e., to satisfy the Madon-Boudart criterion). FIG. 8 shows that Ti-FAU, Ti-BEA, and Ti-FAU all possess nearly indistinguishable dependencies on the concentrations of C₈H₈ (FIG. 8a) and H₂O₂ (FIG. 8b) despite significant differences between the topologies of these silicate frameworks.

All Ti-based catalysts exhibit two kinetic regimes that differ in how epoxidation rates depend on the concentrations of reactants. At low [C₈H₈]:[H₂O₂] (<1), turnover rates increase linearly with [C₈H₈] and do not vary with [H₂O₂] (when [H₂O₂] is >5-10⁻³ M), which suggests that active sites are saturated with reactive species derived from H₂O₂ (e.g., Ti—OOH). At low values of [H₂O₂] (<5-10⁻³ M), turnover rates over Ti-BEA and Ti-FAU show a first-order dependence on both [C₈H₈] and [H₂O₂], which suggests that active sites are saturated with solvent molecules, rather than an intermediate derived from the reactants. At high [C₈H₈]:[H₂O₂](>10), turnover rates are independent of [C₈H₈], indicating that the identity of the most abundant reactive intermediate (MARI) under these conditions is derived from C₈H₈ (e.g., styrene oxide; C₈H₈O). The dependence of C₈H₈O formation on reactant concentrations are identical with prior findings within our group for the epoxidation of cyclohexene, styrene, 1-octene, and sulfoxidation of 2,5-dimethylthiophene over groups 4 and 5-substituted zeolite BEA.

FIG. 7b shows a series of elementary steps that account for the measured effects of [C₈H₈] and [H₂O₂] on the rates of C₈H₈O formation. This proposed catalytic cycle involves the quasi-equilibrated adsorption of C₈H₈ (step 1) and H₂O₂ (step 2) followed by the irreversible activation of H₂O₂ (step 3) to form Ti—OOH surface intermediates. These Ti—OOH intermediates then react with C₈H₈ (step 4) or H₂O₂ (step 6) through rate-determining processes to form Ti-bound C₈H₈O or H₂O₂-decomposition products, respectively. Finally, C₈H₈O molecules desorption is quasi-equilibrated and reforms the Ti active site. Rates of C₈H₈O formation (r_{E}) are given by

r_{E}=k₄[C₈H₈][Ti—OOH] (3)

where kᵢ is the rate constant for step I in FIG. 7b and [Ti—OOH] is the number of Ti—OOH surface intermediates. Application of the pseudo steady-state hypothesis to Ti—OOH surface intermediates, combined with a site balance over all possible configurations for surface intermediates bound to Ti active sites, yields

$$\frac{{r}_{E}}{[ L ]} = \frac{\frac{{k}_{3} {k}_{4} {K}_{2} [ {C}_{8} {H}_{8} ] [ {H}_{2} {O}_{2} ]}{{k}_{4} [ {C}_{8} {H}_{8} ] + {k}_{6} [ {H}_{2} {O}_{2} ]}}{\begin{matrix}1 + {K}_{1} [ {C}_{8} {H}_{8} ] + {K}_{2} [ {H}_{2} {O}_{2} ] + \\ \frac{{k}_{3} {K}_{2} [ {H}_{2} {O}_{2} ]}{{k}_{4} [ {C}_{8} {H}_{8} ] + {k}_{6} [ {H}_{2} {O}_{2} ]} + \frac{[ {C}_{8} {H}_{8} O ]}{{K}_{5}}\end{matrix}} \qquad ( 4 )$$

where Kᵢ is the equilibrium constant for step I, [L] is the total number of Ti atoms loaded into the reactor, and the five terms within the denominator correspond to Ti active sites that are occupied by solvent molecules, adsorbed C₈H₈, adsorbed H₂O₂, Ti—OOH intermediates, and adsorbed C₈H₈O, respectively.

Reaction conditions where turnover rates depend linearly on [C₈H₈] and are independent of [H₂O₂] result in active sites that are saturated with Ti—OOH intermediates and reduces equation 4 to yield

$$\frac{{r}_{E}}{[ L ]} = {k}_{4} [ {C}_{8} {H}_{8} ] \qquad ( 5 )$$

which matches the experimental observations within FIG. 8 at low [C₈H₈]:[H₂O₂]. Equation 4 reproduces the measured dependence on [C₈H₈] at high [C₈H₈]:[H₂O₂] when two conditions are met. First, the formation of appreciable concentrations of C₈H₈O results in the competitive adsorption of epoxide products, which has been observed for the binding of epoxide products to Lewis acidic Ti atoms within Ti-BEA and TS-1. Second, at high [C₈H₈], values of k₄[C₈H₈] become much greater than k₆[H₂O₂] which reduces equation 4 to

$$\frac{{r}_{E}}{[ L ]} = \frac{{k}_{3} {K}_{2} {K}_{5} [ {H}_{2} {O}_{2} ]}{[ {C}_{8} {H}_{8} O ]} \qquad ( 6 )$$

Equation 6 is consistent with the independence of epoxidation turnover rates on [C₈H₈] at high [C₈H₈]:[H₂O₂] within FIG. 8a. Despite the indistinguishable mechanisms between Ti-FAU, Ti-BEA, and Ti—SiO₂, there are significant differences in the magnitude of the rates of epoxidation (e.g., a factor of ˜10 difference between Ti-FAU and Ti—SiO₂). To understand the origin of these differences, equitable comparisons of turnover rates and apparent activation enthalpies and entropies must be made at conditions that result in comparable coverages of surface intermediates.

Thermochemical Analysis Shows Transition State Stabilization. Transition state theory postulates that the rate of reaction depends on the stability of an activated complex (i.e., a transition state) relative to the stability of the stable intermediate immediately preceding it along a reaction trajectory (Scheme 1). In the context of alkene epoxidation, Ti—OOH—C₈H₈^{‡} represents the transition state for C₈H₈ epoxidation which forms transiently upon reaction between Ti—OOH reactive intermediates with proximate C₈H₈.

$$Ti —OOH + {C}_{8} {H}_{8} {Ti —OOH—C}_{8} {H}_{8}^{‡}$$

Within the tenets of transition state theory, turnover rates for C₈H₈ epoxidation, under conditions that result in Ti—OOH MARI, are given by

$$\frac{{r}_{E}}{[ L ]} = \frac{{k}_{B} T}{h} {K}_{E}^{‡} [ {C}_{8} {H}_{8} ] \qquad ( 7 )$$

where k_{B} is the Boltzmann constant, h is Planck's constant, T is the absolute temperature, and K_{E}‡ is the transition state equilibrium constant for epoxidation. K_{E}^{‡} depends on the thermodynamic stability of the transition state relative to the reference state and takes the form

$${K}_{E}^{‡} = e \frac{- ( Δ {H}_{A p p}^{‡} - T Δ {S}_{A p p}^{‡} )}{R T} \qquad ( 8 )$$

where ΔH_{App}^{‡} and ΔS_{App}^{‡} are the apparent activation enthalpy and entropy for epoxidation, respectively.

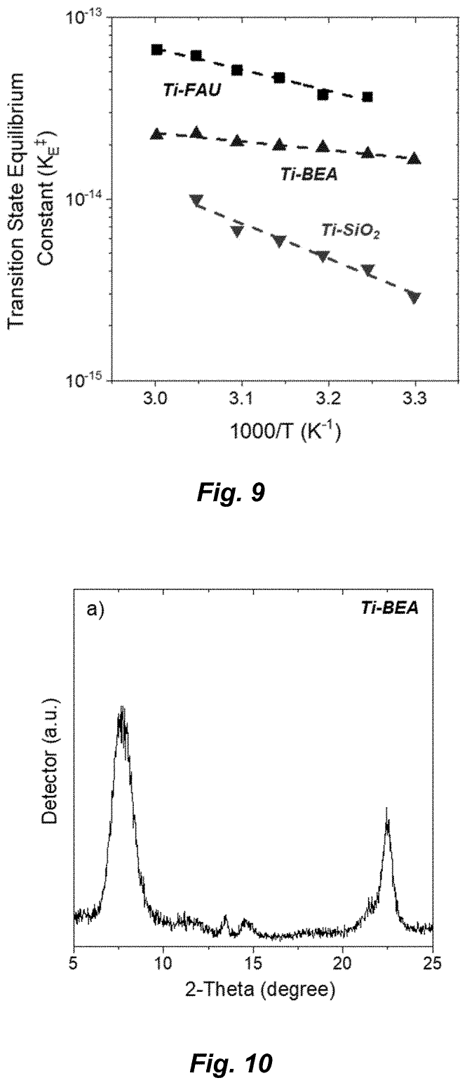

FIG. 9 shows K_{E}I as a function of inverse temperature for Ti-FAU, Ti-BEA, and Ti—SiO₂ used to determine the ΔH_{App}^{‡} and ΔS_{App}^{‡} Table 2 shows ΔH_{App}^{‡} and ΔS_{App}^{‡} both C₈H₈ epoxidation obtained under reaction conditions that result in Ti—OOH saturated surfaces and in the absence of mass-transfer restrictions. Values of ΔH_{App}^{‡} increase in the order of Ti-BEA <Ti-FAU <Ti-SiO₂, which suggests that the smaller pores of the *BEA framework (˜0.65 nm) enthalpically stabilize the transition state for C₈H₈ epoxidation relative to FAU (1.2 nm) and SiO₂ (5.4 nm). This stabilization results from the solvation of Ti—OOH—C₈H₈^{‡} from the pore walls of the silicate support. The great extent of confinement of C₈H₈ epoxidation transition states within the voids of the Ti-catalyst, however, results in a disproportionate entropy of activation, where epoxidation within Ti-BEA incurs the greatest entropic loss, followed by Ti-FAU and Ti—SiO₂. This entropy of activation primarily reflects the loss of translational motion from fluid-phase C₈H₈ upon adsorption and formation of Ti—OOH—C₈H₈^{‡}. For example, within Ti—SiO₂, the mesopore surrounding the Ti active site allows for the greatest flexibility of the C₈H₈ transition state, which results in the smallest loss of entropy among these three catalysts.

TABLE 2
Apparent activation enthalpies and entropies for C₈H₈
epoxidation over Ti—FAU, Ti—BEA, and Ti—FAU under
reaction conditions that result in a Ti—OOH saturated surface.

| | ΔH_{App}^{‡} | ΔS_{App}^{‡} |
|---|---|---|
| Catalyst | (kJ mol⁻¹) | (J mol⁻¹ K⁻¹) |
| Ti—FAU | 22 ± 2 | −185 ± 15 |
| Ti—BEA | 9 ± 2 | −234 ± 20 |
| Ti—SiO₂ | 37 ± 4 | −155 ± 15 |

Epoxidation within Ti-FAU results in intermediate values of ΔH_{App}^{‡} and ΔS_{App}^{‡}, which results in favorable enthalpic stabilization due to the surrounding supercage relative to Ti—SiO₂; yet, provides enough flexibility for Ti—OOH—C₈H₈‡ as compared to Ti-BEA. Ti—SiO₂ possesses a greater dependence on temperature than Ti-FAU, which suggests that at a high enough temperature the rates should be greater on Ti—SiO₂. The isokinetic point (i.e., the temperature at which the rates of C₈H₈ epoxidation are equal) between Ti-FAU and Ti-FAU, however, lies outside the solvent temperature window (CH₃CN has a boiling point of 82° C. at atmospheric pressure), such that Ti-FAU will always possess a greater rate than Ti—SiO₂ within these types of reactors. Additional evidence for the selective stabilization of bulky aromatic transition states (i.e., for 2,4-dimethylstyrene) within Ti-FAU relative to other Ti-silicates is provided in Example 5 below.

Conclusions. Multiple treatments of Al-FAU (Si:Al=15) in HNO₃ removes nearly all of the Al atoms to produce siliceous FAU (Si:Al >900). These treatments are necessary to remove the adventitious Al atoms, that may act as deleterious sites during zeolite catalysis (e.g., within sugar isomerization, alcohol upgrading, alkene epoxidation). The liquid-phase grafting of metal chlorides and alkoxides leads to the isomorphic substitution of metal (M=Ti, Nb, Ta, Sn) atoms into the framework of FAU. Ti-FAU catalysts efficiently activate H₂O₂ to form Ti—OOH intermediates that are active for alkene epoxidation. In the case of styrene epoxidation, Ti-FAU possesses rates of epoxidation that are greater than Ti-BEA and Ti—SiO₂ by factors of 2 and 7, respectively. Rates of H₂O₂ decomposition, however, are invariant with the characteristic pore diameter of the Ti-silicate catalyst. Differences in catalysis are not due to differences in the mechanism for epoxidation; yet, reflect differences in the stability of the transition states for C₈H₈ epoxidation. Specifically, C₈H₈ epoxidation transition states are enthalpically stabilized within Ti-FAU relative to Ti—SiO₂ and also possess greater entropic freedom than within Ti-BEA, which results in the lowest free energies within the bounds of solvent stability. The work presented here serves as an exemplary example for how zeolite framework topology can be chosen to selectively stabilize desired surface intermediates. The synthetic protocols established here will enable the design of new materials in the quest for the rational development of catalysts.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. FAU Dealumination

Heteroatom-substituted FAU (M-FAU) were prepared through the post-synthetic modification of W-form Al-FAU (Zeolyst, CBV 720; Si:Al 15). Al-FAU was treated in HNO₃ (Macron Chemicals, 68-70 wt. %, 20 cm³ g_{Al-FAU}⁻¹) at reflux for 18 hours with the intent to remove framework Al by forming soluble Al(NO₃)₃. The solids were recovered by vacuum filtration, washed with additional HNO₃ (5 cm³ g_{Al-FAU}⁻¹), and deionized H₂O (17.8 MΩ cm; 25 cm³ g_{Al-FAU}⁻¹). Note that concentrated HNO₃ can easily cause chemical burns and should be handled carefully. These washed solids were then dried at 823 K (5 K min⁻¹) for 6 h in flowing air (Airgas, Ultra-zero grade; 100 cm³ min⁻¹) to produce a partially-dealuminated FAU with a Si:Al of ˜200 (estimated by energy dispersive X-ray fluorescence, see below). The partial dealumination may result from the redeposition of Al into the FAU framework during filtration at room temperature. To further remove Al atoms, these dried solids were then subjected to a second dealumination sequence identical to that described above to produce Si-FAU with a Si:Al greater than 900.

### Example 2. Heteroatom Incorporation

Ti and Sn atoms were incorporated into the FAU framework through the liquid-phase grafting of TiCl₄ (Sigma-Aldrich, 99.9%) or SnCl₄.5H₂O (Sigma-Aldrich, 98%) in dichloromethane (DCM, Fisher Chemicals, Certified ACS Stabilized, 25 cm³ g_{Si-FAU}⁻¹). Nb- and Ta-substituted FAU were prepared by refluxing Nb(OEt)₅ (Sigma Aldrich, 99.95%) or Ta(OEt)₅ (Sigma-Aldrich, 99.98%) in isopropanol (Fisher Chemical, Optima, 25 cm³ g_{Si-FAU}⁻¹). In all cases, the suspensions containing Si-FAU and MCl₄ or M(OEt)₅ were kept under an argon atmosphere using standard Schlenk technique for at least 6 h. Prior to introduction of the solvent and the MClₓ or M(OEt)₅ precursor, the Si-FAU was dehydrated at 473 K under vacuum (<5 Pa) for 3 h to desorb any residual H₂O that may hydrolyze the metal precursors. Dehydration of Si-FAU is particularly important when using the TiCl₄ precursor, which readily hydrolyzes to form oligomeric and bulk TiOₓ aggregates. The solvent and other volatile components were removed via rotary evaporation and the recovered solids were heated in flowing air (100 cm³ min⁻¹) to 823 K at 5 K min⁻¹ and held for 6 h to yield bright, white-colored solids in all cases.

### Example 3. Preparation of Other Modified Zeolites

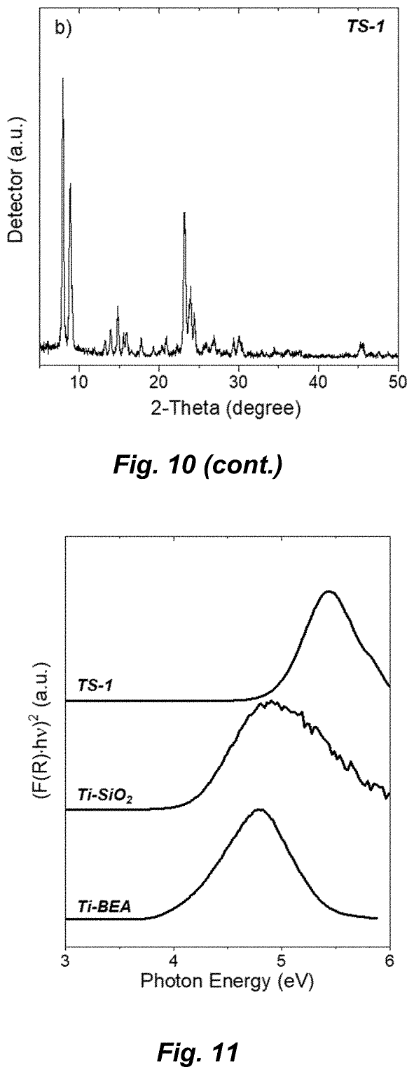

Titanium substituted BEA (Ti-BEA) and Ti-grafted SiO₂ (Ti—SiO₂) materials were used within a previous study (ACS Catal., 2018, 8, 2995), which presents the detailed chemical and physical characterization of these materials. The titanium silicalite-1 (TS-1) material was synthesized according to the recommended procedure from the International Zeolite Association's Synthesis Commission to contain 0.3 wt. % Ti. The relevant characterization data is presented within the Supporting Information (FIG. 10 and FIG. 11).

Briefly, Ti-BEA was prepared by the post-synthetic modification of commercial Al-BEA (Zeolyst, CP814E). Al-BEA was contacted with HNO₃ at reflux for 18 h with the intent of forming soluble Al(NO₃)₃. The solids were recovered by vacuum filtration and washed thoroughly with H₂O prior to dehydration at 823 K (6 h; 5 K min⁻¹) in flowing air (100 cm³ min⁻¹), which produced Si-BEA (Si:Al >1200). Ti atoms were incorporated into Si-BEA through the liquid-phase incorporation of TiCl₄ in DCM at reflux. Volatile components were removed via rotary evaporation and recovered solids were treated at 823 K (5 K min⁻¹) for 6 h in flowing air (100 cm³ min⁻¹) to produce Ti-BEA.

Ti—SiO₂ was synthesized through the grafting of titanium 1,3-dimethoxy-tert-butylcalix[4]arene (Ti-dmCalix) onto SiO₂ (Selecto Scientific, 32-62 m particle size, 5.4 nm pore diameter) through reflux in toluene in an Ar atmosphere. Prior to grafting, SiO₂ was dehydroxylated at 573 K under vacuum (<5 Pa) for 10 h to produce isolated SiOH and minimize any Ti—O—Ti oligomer formation. Ti-dmCalix-grafted SiO₂ was recovered by vacuum filtration and treated at 823 K (5 K min⁻¹) for 6 h in air (100 cm³ min⁻¹) to produce Ti—SiO₂.

TS-1 was synthesized hydrothermally in hydroxide media. In short, 150 mg of titanium (IV) butoxide (TBOT; Sigma-Aldrich, 97%) was dissolved in 27.7 g of tetraethylorthosilicate (TEOS; Sigma-Aldrich, 98%) in a polypropylene container to form a homogeneous solution and was subsequently cooled to 273 K. Separately, a mixture of 28.7 g of tetrapropylammonium hydroxide (TPAOH; Sachem, 40% in H₂O) and 50.5 g of H₂O was cooled to 273 K and was slowly added (over ˜1 min) to the solution of TBOT and TEOS, which yielded a biphasic mixture. This solution was then warmed to 298 K and stirred for 12 h to produce a homogeneous solution, which indicates complete hydrolysis of the TBOT and TEOS. The cover was then removed to evaporate the ethanol and butanol formed through hydrolysis and produce a solution with the approximate composition of 1 Si:0.0033 Ti:0.43 TPAOH:28.3 H₂O. This solution was then loaded into a Teflon-lined stainless-steel autoclave (Parr instruments, 125 cm³) that contained 5% (relative to SiO₂ in the gel) TS-1 from a previous synthesis. This autoclave was heated to 443 K while rotating (30 rpm) in a convection oven for 3 days. The resulting solids were recovered by centrifugation, washed with H₂O, and dried for 16 h at 373 K. The dried solids were then heated in flowing air (100 cm³ min⁻¹) at 823 K (1 K min⁻¹) for 10 h to produce TS-1.

### Example 4. Catalyst Characterization

The metal contents of all M-FAU were determined using energy dispersive X-ray fluorescence. Finely-ground M-FAU samples were loaded into a polypropylene sample holder (2.45 cm aperture) which was sealed with ultralene film. These samples were loaded into a spectrometer (Shimadzu, EDX-7000) whose sample chamber was purged with He (Airgas, Ultra-zero grade) prior to measurement. Spectra were obtained between 0 and 30 keV (500 scans), and the relative intensities of the element-specific fluorescence features and their associated calibration factors were used to determine the percent, by mass, of each element within the sample.

The crystallinity and contraction/expansion of the FAU framework was measured through X-ray diffraction. Samples were loaded onto a polypropylene holder and X-ray diffractograms were collected on a diffractometer (Siemens/Bruker, D5000) with Cu Kα radiation (0.15418 nm) under ambient conditions.

The surface area and pore-size distributions of M-FAU were determined by N₂ adsorption. Gas-phase N₂ adsorption isotherms (77 K) were collected on a volumetric adsorption instrument (Micromeritics, 3Flex). Samples (50-100 mg) were pelletized and sieved to retain particles between 250 and 500 μm in diameter. These samples were degassed by heated under vacuum (<0.7 Pa, 673 K) for 6 h prior to adsorption measurements. Pore size distributions were determined from N₂ adsorption isotherms using a cylindrical pore model with non-local density functional theory (NLDFT) in the 3Flex software.

The presence of highly-disperse M atoms (and absence of bulk or oligomeric MOₓ domains) was inferred by the band edge energies, which were measured using diffuse reflectance UV-vis spectroscopy. Total reflectance spectra were measured under ambient conditions using a diffuse-reflectance accessory (Harrick, Cricket) with a UV-Vis-NIR spectrophotometer (Agilent, CARY 5). Prior to measurement, samples were intimately mixed with magnesium oxide (MgO; Sigma-Aldrich, 99.995%) in a 1:10 ratio by mass.

Infrared (IR) spectra of adsorbed pyridine (Sigma-Aldrich, 99.8%) were used to confirm the presence of Lewis acid sites within M-FAU and to detect Brønsted acid sites associated with remaining framework Al atoms. See FIG. 16. IR spectra (128 scans, 4 cm⁻¹) were obtained at equilibrium pyridine coverages using a custom-built temperature-controlled transmission cell coupled to a Fourier-transform IR spectrometer (Bruker, Tensor 37) with a liquid N₂-cooled HgCdTe detector. Thin catalyst pellets (45 mg) were loaded into the transmission cell, which was configured with CaF₂ windows and connected to a gas manifold equipped with a liquid-injection port. All materials were first heated to 573 K (10 K min⁻¹) and held for >2 h in flowing He (50 cm³ min⁻¹) to desorb any volatile compounds. Pyridine was introduced using a syringe pump (KD Scientific, Legato 100) and vaporized in the gas-transfer lines into a stream of He (50 cm³ min⁻¹) to contact the M-FAU pellets.

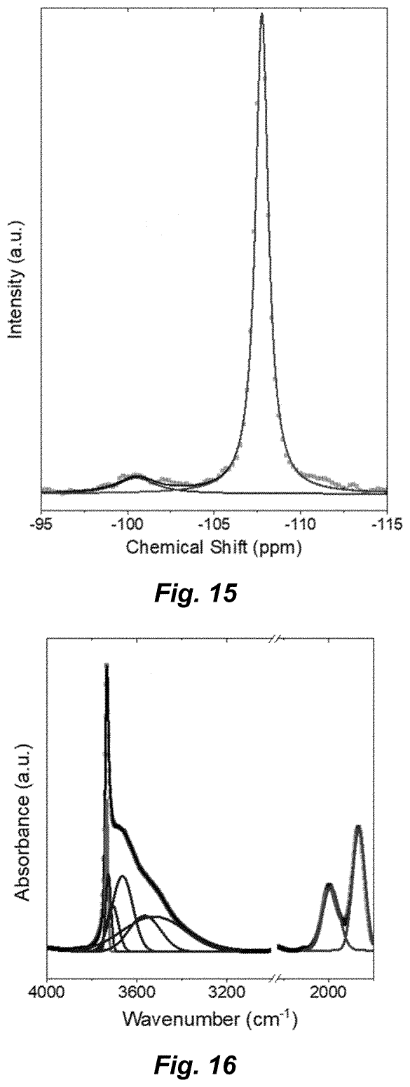

The fraction of Si atoms that exist as Si(OSi)₃OH (ϕ_{NMR}) within M-FAU samples were determined using ²⁹Si magic angle spinning-nuclear magnetic resonance (MAS-NMR) spectroscopy. MAS-NMR spectra (4,000 scans) were collected on a spectrometer (Varian, Unity Inova 300 MHz; 7.05 T), operating at 59.6 MHz Larmor frequency, equipped with a 4 mm MAS probe (Varian-Chemagnetics, double-resonance HX, APEX) under ambient conditions. M-FAU samples (˜70 mg) were loaded into 4.0 mm outer diameter zirconia rotors that were spun at 10 kHz. Powdered octakis(trimethylsiloxy)silsesquioxane (Q₈M₈) was used for ²⁹Si chemical shift referencing (Q₈M₈ has a chemical shift of 11.45 ppm relative to tetramethylsilane (TMS) at 0 ppm). See FIG. 15. Pulse width calibration was performed on Si-FAU, which yielded a 90° pulse width of 1.5 s. The recycling delay (di) for SI-FAU was varied between 5 and 15 s to determine how ϕ_{NMR} changed with d₁; a d₁ of 10 s was used for all M-FAU as NMR was identical for a d₁ of 10 and 15 s.

### Example 5. Measurement of Epoxidation Rates

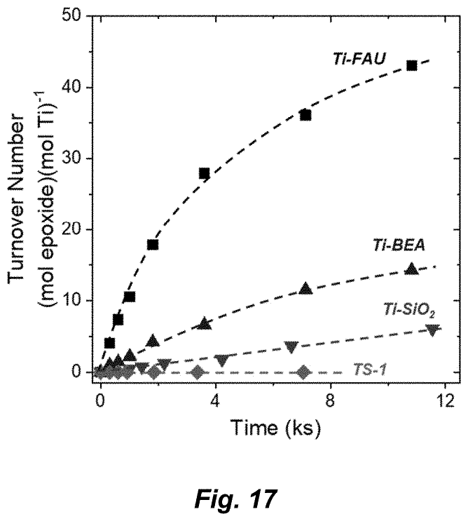

For all kinetic measurements, a Ti-FAU sample with 0.3 wt. % Ti atoms was used to avoid artifacts from internal mass-transfer restrictions. Rates of styrene (C₈H₈; Sigma-Aldrich, 99%) and 2,4-dimethylstyrene (C₁₀H₁₂; Sigma-Aldrich, 97%) epoxidation were measured in batch reactors (100 cm³, three-neck round bottom flasks) equipped with reflux condensers to minimize evaporative losses. See FIG. 17. Solutions of C₈H₈ or C₁₀H₁₂ and H₂O₂ (Fisher; 30 wt. % in H₂O) with Benzene (internal standard; Sigma-Aldrich, >99% thiophene-free) in acetonitrile (Fisher Chemicals, HPLC grade) was heated to the desired temperature (308-348 K) while stirring at 700 rpm. Epoxidation was initiated by the introduction of Ti-FAU and small aliquots were extracted as a function of time through a 0.22 m syringe filter. The concentrations of all organic components within these aliquots were determined using a gas chromatograph (HP, 5890 Series A) equipped with a flame-ionization detector. The concentration of H₂O₂ in each aliquot was determined by colorimetric titration using aqueous CuSO₄ (8.3 mM, Sigma-Aldrich, >98%) indicator with neocuproine (12 mM, Sigma-Aldrich, >98%) and ethanol (25% v/v, Decon Laboratories, 100%). Notably, styrene oxidation results in the formation of styrene oxide (C₈H₈O) and phenylacetaldehyde. Extrapolation of the selectivity towards each of these species to the limit of zero conversion shows that phenylacetaldehyde forms by C₈H₈O isomerization. Here, the combined concentrations of C₈H₈O and phenylacetaldehyde were used to calculate the turnover rates for epoxidation reactions.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A modified zeolite comprising dealuminated faujasite that has a crystalline framework and micropores, wherein a metal heteroatom (M) is integrated into a dealuminated node of the dealuminated faujasite via a M-O—Si linkage;
wherein the modified zeolite has a silicon to aluminum (Si:Al) mole ratio of about 200 or greater and a silicon to integrated metal (Si:M) mole ratio of about 15 or greater.

2. The modified zeolite of claim 1 wherein the Si:Al mole ratio is about 500 or greater.

3. The modified zeolite of claim 1 wherein the Si:Al mole ratio is about 900 to about 2000.

4. The modified zeolite of claim 1 wherein the Si:M mole ratio is about 30 to about 50.

5. The modified zeolite of claim 1 wherein the integrated metal heteroatom is an early transition metal or metalloid.

6. The modified zeolite of claim 1 wherein the integrated metal heteroatom is titanium, niobium, tantalum, zirconium, hafnium, molybdenum, tungsten, tin, germanium, or a combination thereof.

7. The modified zeolite of claim 1 wherein the integrated metal heteroatom is titanium.

8. The modified zeolite of claim 1 wherein the integrated metal heteroatom is exposed at the surface of the micropores.

9. The modified zeolite of claim 1 wherein the micropores have an average diameter of about 1 nm to about 2 nm.

10. A method for forming the modified zeolite according to claim 1 comprising:
(a) contacting unmodified faujasite (FAU) and a mineral acid at a reflux temperature to form a dealuminated faujasite comprising dealuminated nodes;
(b) filtering, rinsing and drying the dealuminated faujasite; and
(c) repeating steps a) and b) optionally one or more times;
wherein the modified zeolite is thereby formed.

11. The method of claim 10 further comprising integrating a metal heteroatom into a dealuminated node of the dealuminated faujasite.

12. The method of claim 11 wherein the metal heteroatom is integrated by anhydrous liquid-phase grafting.

13. The method of claim 10 wherein the mineral acid is nitric acid, hydrochloric acid, sulfuric acid, or a combination thereof.

14. A method for catalyzing an oxidation reaction comprising:
contacting the modified zeolite catalyst according to claim 1, an oxidizing agent and a substrate under suitable catalytic reaction conditions;
wherein the substrate and oxidizing agent have a sufficient size to enter a micropore of the modified zeolite for catalysis, wherein the substrate undergoes an oxidation reaction at an integrated metal heteroatom inside the micropore that is accessible for catalyzing the oxidation reaction.

15. The method of claim 14 wherein the integrated metal heteroatom is titanium.

16. The method of claim 14 wherein the oxidizing agent is a peroxide.

17. The method of claim 14 wherein the substrate is an olefin.

18. The method of claim 14 wherein the catalyzed oxidation reaction has a turnover rate at least two-times greater than a corresponding oxidation reaction catalyzed by other modified zeolites that are not faujasite.

19. The method of claim 14 wherein the substrate is an olefin, the oxidizing agent is a peroxide, the integrated metal heteroatom is titanium, the micropores have diameters of about 1 nm to about 2 nm, and the Si:Al mole ratio is about 900 to about 2000.

20. The method of claim 14 wherein the suitable catalytic reaction conditions comprise a polar aprotic solvent and a reaction temperature of about −10° C. to about 80° C.
